(19) United States
(12) Patent Application Publication
(10) Pub. No.: US 2025/0281117 A1
(43) Pub. Date: Sep. 11, 2025

(54) MEDICAL SENSOR, AND SENSOR SUPPORTING MEMBER

(71) Applicant: NIHON KOHDEN CORPORATION, Tokyo (JP)

(72) Inventor: Takahiro ABE, Saitama (JP)

(73) Assignee: NIHON KOHDEN CORPORATION, Tokyo (JP)

(21) Appl. No.: 19/068,898

(22) Filed: Mar. 3, 2025

(30) Foreign Application Priority Data
Mar. 11, 2024 (JP) 2024-037120

(51) Int. Cl. A61B 5/00; A61B 5/1455

(52) U.S. Cl. CPC A61B 5/6832; A61B 5/14552; A61B 2560/0462

(57) ABSTRACT

At least one light emitter is configured to emit light. At least one light detector is configured to output a signal corresponding to an amount of incident light. A supporting member is adapted to be attached to a body of a subject. The supporting member includes a first supporting portion, a second supporting portion and a third supporting portion. The first supporting portion supports and covers the light emitter. The second supporting portion supports and covers the light detector. The third supporting portion is located between the first supporting portion and the second supporting portion. At least one of the first supporting portion, the second supporting portion and the third supporting portion includes a portion with higher flexibility than an adjacent portion thereof.

### CROSS-REFERENCE TO RELATED APPLICATION

The present application is based on Japanese Patent Application No. 2024-037120 filed on Mar. 11, 2024, the entire contents of which are incorporated herein by reference.

### BACKGROUND

The present disclosure relates to a medical sensor provided with a light emitter that emits light, and a light detector that outputs a signal corresponding to an amount of incident light. The present disclosure also relates to a sensor supporting member for attaching the medical sensor to a body of a subject.

Japanese Patent Publication No. 2020-150996A discloses, as an example of a medical sensor, a sensor adapted to be attached to a fingertip of a subject to calculate a transcutaneous arterial oxygen saturation (SpO2) of the subject. The sensor includes a light emitter and a light detector. The light emitter emits light toward a tissue of the fingertip. The light having passed through the tissue is received by the light detector. The light detector outputs a signal corresponding to intensity of the detected light. The sensor includes a supporting member adapted to be wrapped around the fingertip. The supporting member supports the light emitter and the light detector. The signal that is outputted from the light detector is transmitted through a cable to a device that calculates SpO2.

### SUMMARY

It is demanded to improve the convenience of a medical sensor configured such that a light emitter and a light detector are attached to a subject's body using a supporting member.

A first illustrative aspect of the present disclosure may provide a medical sensor, comprising:
at least one light emitter configured to emit light;
at least one light detector configured to output a signal corresponding to an amount of incident light; and
a supporting member adapted to be attached to a body of a subject,
wherein the supporting member includes:
   a first supporting portion supporting and covering the light emitter;
   a second supporting portion supporting and covering the light detector; and
   a third supporting portion located between the first supporting portion and the second supporting portion; and
wherein at least one of the first supporting portion, the second supporting portion and the third supporting portion includes a portion with higher flexibility than an adjacent portion thereof.

A second illustrative aspect of the present disclosure may provide a sensor supporting member adapted to attach, to a body of a subject, a medical sensor provided with at least one light emitter configured to emit light and at least one light detector configured to output a signal corresponding to an amount of incident light, the sensor supporting member comprising:
a first supporting portion supporting and covering the light emitter;
a second supporting portion supporting and covering the light detector; and
a third supporting portion located between the first supporting portion and the second supporting portion,
wherein at least one of the first supporting portion, the second supporting portion and the third supporting portion includes a portion with higher flexibility than an adjacent portion thereof.

The first supporting portion is a portion that supports and covers the light emitter. According to the configuration of each of the first and second illustrative aspects, elongation of the first supporting portion by covering the light emitter is promoted, and generation of a stress in the supporting member, and misalignment of the light emitter due to the stress can be suppressed.

The second supporting portion is a portion that supports the light detector while covering the light detector. According to the configuration of each of the first and second illustrative aspects, elongation of the second supporting portion due to the coverage of the light detector is promoted, so that it is possible to suppress generation of a stress in the supporting member as well as the misalignment of the light emitter due to such stress.

The third supporting portion is a portion that is to be bent in order to cause the light emitter and the light detector to face each other. According to the configuration of each of the first and second illustrative aspects, not only the supporting member can be bent with a smaller force, but also the maintainability of the bent state is enhanced. As a result, it is possible to suppress misalignment of the light emitter and the light detector after the medical sensor is fixed to the body of the subject.

Therefore, it is possible to improve the convenience of the medical sensor configured such that the light emitter and the light detector are attached to the body of the subject with the supporting member.

A third illustrative aspect of the present disclosure may provide a medical sensor, comprising:
at least one light emitter configured to emit light;
at least one light detector configured to output a signal corresponding to an amount of incident light; and
a supporting member adapted to be attached to a body of a subject,
wherein the supporting member includes:
   a first retaining portion having a first adhesive face adapted to be adhered to the body; and
   a second retaining portion having a second adhesive face adapted to be adhered to a surface of the first retaining portion while covering the surface,
wherein at least a part of the surface includes a portion with lower adhesiveness than an adjacent portion thereof.

A fourth illustrative aspect of the present disclosure may provide a sensor supporting member adapted to attach, to a body of a subject, a medical sensor provided with at least one light configured to emit light and at least one light detector configured to output a signal corresponding to an amount of incident light, the sensor supporting member comprising:
a first retaining portion having a first adhesive face adapted to be adhered to the body; and
a second retaining portion having a second adhesive face adapted to be adhered to a surface of the first retaining portion while covering the surface,
wherein at least a part of the surface includes a portion with lower adhesiveness than an adjacent portion thereof.

When the medical sensor is fixed to the body of the subject, the second adhesive face is adhered to the surface of the first retaining portion. Since a portion having low adhesiveness is formed on the surface, the peelability of the second retaining portion from the first retaining portion can be enhanced. As a result, it is possible to facilitate the operation of re-attaching the sensor in order to, for example, optimize the arrangement of the light emitter and the light detector. Therefore, it is possible to improve the convenience of the medical sensor configured such that the light emitter and the light detector are attached to the body of the subject with the supporting member.

### DESCRIPTION OF THE DRAWINGS

FIG. 1 illustrates an appearance of a front face side of a sensor according to an exemplary embodiment.

FIG. 2 illustrates an appearance of a back face side of the sensor of FIG. 1.

FIG. 3 illustrates how to attach the sensor of FIG. 1 to a finger of a subject.

FIG. 4 illustrates how to attach the sensor of FIG. 1 to the finger of the subject.

FIG. 5 illustrates how to attach the sensor of FIG. 1 to the finger of the subject.

FIG. 6 illustrates an exemplary cross-sectional structure of a supporting member in a region VI illustrated in FIG. 1.

FIG. 7 illustrates another exemplary cross-sectional structure of the supporting member in the region VI illustrated in FIG. 1.

FIG. 8 illustrates an appearance of a front face side of a sensor according to another exemplary configuration.

FIG. 9 illustrates an exemplary cross-sectional structure of a supporting member in a region IX illustrated in FIG. 8.

FIG. 10 illustrates another exemplary cross-sectional structure of the supporting member in the region IX illustrated in FIG. 8.

FIG. 11 illustrates an appearance of a sensor according to another exemplary configuration.

### DESCRIPTION OF EMBODIMENTS

Examples of embodiments will be described below in detail with reference to the accompanying drawings. In each of the drawings, the scale is appropriately changed in order to make each element as illustrated have a recognizable size.

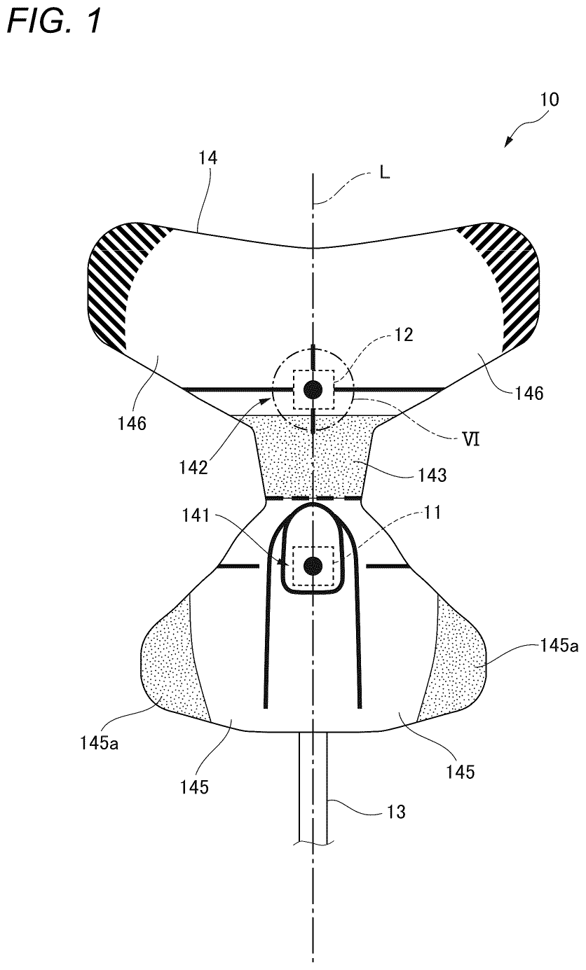

FIG. 1 illustrates an appearance of a sensor 10 according to an exemplary embodiment. The sensor 10 is attached to a fingertip of a subject to calculate a blood light absorber concentration. The sensor 10 is an example of a medical sensor. The fingertip is an example of a body of a subject. Examples of the blood light absorber concentration include a percutaneous arterial oxygen saturation (SpO2).

The sensor 10 includes at least one light emitter 11. The light emitter 11 is a semiconductor light emitter that emits light including a prescribed wavelength. Examples of the semiconductor light emitter include a light emitting diode and a laser diode. The number of light emitter 11 and the value of the wavelength are appropriately determined in accordance with the type of the blood light absorber concentration to be acquired.

The sensor 10 includes at least one light detector 12. The light detector 12 outputs a detection signal corresponding to an amount of light detected on a light detecting face. Examples of the light detector 12 include a photodiode, a phototransistor, and a photoresistor.

The sensor 10 includes a cable 13. The sensor 10 is connected to a pulse photometer (not illustrated) through the cable 13. The cable 13 accommodates a power supply line, a control signal line, and a detection signal line.

The power supply line and the control signal line are connected to a driving circuit (not illustrated) that is connected to the light emitter 11. The power supply line supplies power from the pulse photometer to the driving circuit. The control signal line supplies a control signal transmitted from the pulse photometer to the driving circuit for controlling the activation or deactivation of the light emitter 11.

The power supply line is also connected to the light detector 12. Accordingly, the electric power from the pulse photometer is also supplied to the light detector 12 through the power supply line. The detection signal line electrically connects the light detector 12 and the pulse photometer. Accordingly, the detection signal outputted from the light detector 12 is transmitted to the pulse photometer, and subjected to appropriate processing.

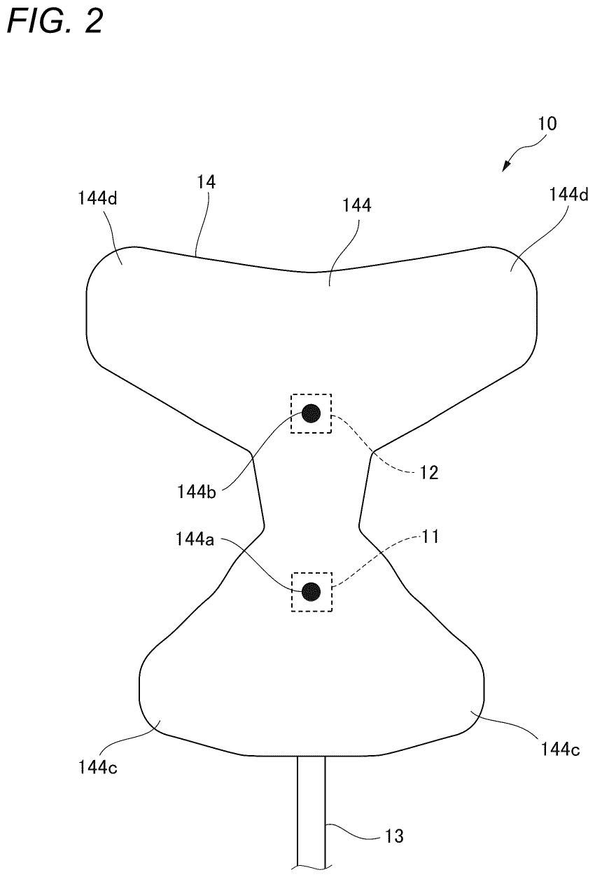

The sensor 10 includes a supporting member 14. FIG. 1 illustrates a front face side of the supporting member 14. FIG. 2 illustrates a back face side of the supporting member 14.

As illustrated in FIG. 1, the supporting member 14 has a first supporting portion 141, a second supporting portion 142, and a third supporting portion 143. The first supporting portion 141 supports and covers the light emitter 11. The second supporting portion 142 supports and covers the light detector 12. The third supporting portion 143 is located between the first supporting portion 141 and the second supporting portion 142.

In the initial state, the back face side of the supporting member 14 is covered with a peelable sheet (not illustrated). When a user peels the sheet upon the usage of the sensor 10, an adhesive face 144 illustrated in FIG. 2 is exposed. The adhesive face 144 has adhesiveness.

The adhesive face 144 includes a first translucent portion 144a and a second translucent portion 144b. The first translucent portion 144a is disposed so as to face the light emitter 11. The second translucent portion 144b is disposed so as to face the light detector 12.

Each of the first translucent portion 144a and the second translucent portion 144b is formed of a material having transparency with respect to the wavelength of the light emitted from the light emitter 11.

A method of attaching the sensor 10 to a finger 20 of a subject will be described with reference to FIGS. 1 to 5.

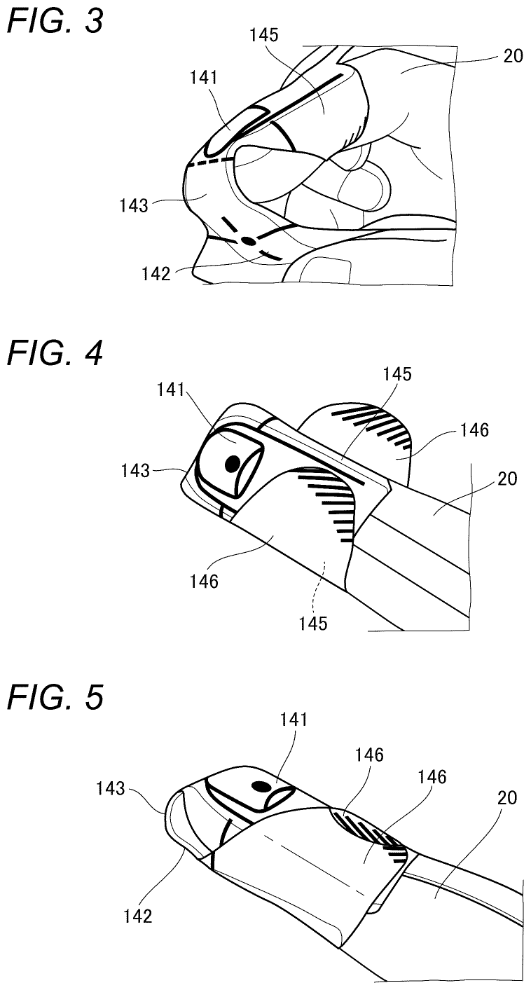

As illustrated in FIG. 3, the first supporting portion 141 is disposed so as to face a nail side of the finger 20 under the condition that the adhesive face 144 of the supporting member 14 is exposed by the peeling of the sheet on the back face side. The adhesive face 144 located on a back face side of the first supporting portion 141 is adhered to the nail side of the finger 20.

As illustrated in FIG. 1, the supporting member 14 includes a first retaining portion 145. The first retaining portion 145 extends leftward and rightward of the first supporting portion 141. As illustrated in FIG. 2, the adhesive face 144 includes a first adhesive face 144c. The first adhesive face 144c is located on a back face side of the first retaining portion 145.

As illustrated in FIG. 3, the first retaining portion 145 is wrapped around the finger. As a result, the first adhesive face 144c is adhered to the finger 20.

Subsequently, the supporting member 14 is bent while the third supporting portion 143 is faced to a tip of the finger 20, whereby the second supporting portion 142 is disposed so as to face a ball side of the finger 20. The adhesive face 144 located on a back face side of the third supporting portion 143 is adhered to the tip of the finger. The adhesive face 144 located on a back face side of the second supporting portion 142 is adhered to the ball side of the finger 20.

As illustrated in FIG. 1, the supporting member 14 includes a second retaining portion 146. The second retaining portion 146 extends leftward and rightward of the second supporting portion 142. As illustrated in FIG. 2, the adhesive face 144 includes a second adhesive face 144d. The second adhesive face 144d is located on a back face side of the second retaining portion 146.

As illustrated in FIGS. 4 and 5, the second retaining portion 146 is wrapped around a portion of the supporting member 14 including the first retaining portion 145. The second adhesive face 144d is adhered to the surface of the first retaining portion 145 while covering the surface. As a result, the retention of the sensor 10 to the finger 20 is finished.

In this state, the light emitter 11 and the light detector 12 are disposed so as to face each other with the finger 20 in between. The light emitted from the light emitter 11 that has passed through the first translucent portion 144a of the supporting member 14 is incident on the tissue of the finger 20. The light that has passed through the tissue and the second translucent portion 144b of the supporting member 14 is incident on the light detector face of the light detector 12. The pulse oximeter performs processing for calculating a light absorber concentration in arterial blood contained in the tissue based on the detection signal outputted from the light detector 12.

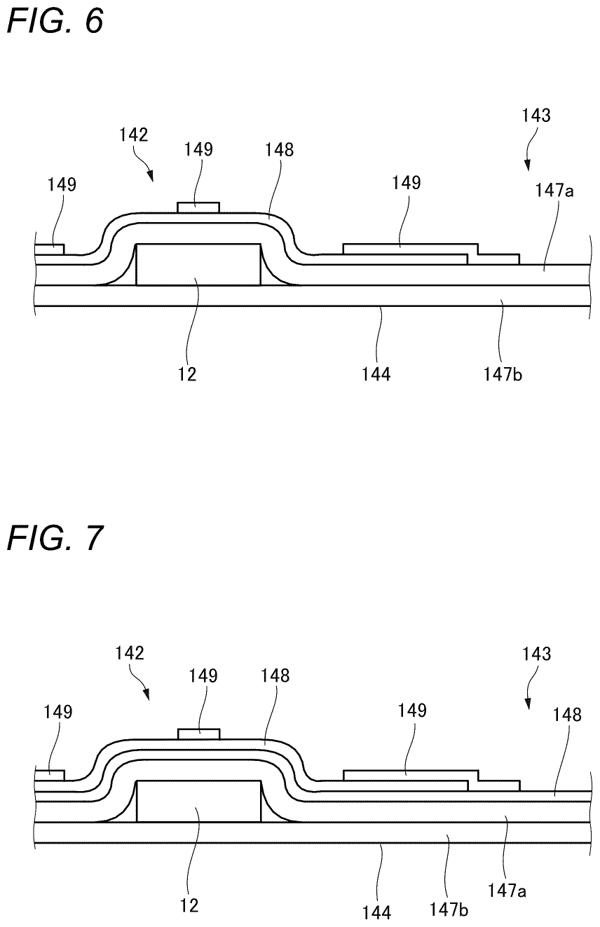

FIG. 6 illustrates a cross-sectional configuration of the supporting member 14 corresponding to a region VI illustrated in FIG. 1. The supporting member 14 includes a first substrate 147a and a second substrate 147b. The first substrate 147a is located on the front face side of the supporting member 14. The second substrate 147b is located on the back face side of the supporting member 14 so as to form the adhesive face 144. The light detector 12 is supported between the first substrate 147a and the second substrate 147b. Although not illustrated, the light emitter 11 is similarly supported between the first substrate 147a and the second substrate 147b.

The supporting member 14 includes a first surface-treated layer 148. The first surface-treated layer 148 has at least one different physical property from the first substrate 147a. In the present exemplary embodiment, the first substrate 147a and the first surface-treated layer 148 have different surface smoothness. Namely, the surface smoothness is an example of the physical property.

As used herein, the term “surface treatment” is meant to include any treatment that is capable of adding another layer having a different physical property to the first substrate 147a. Examples of such a treatment include printing, coating, and adhesion of a film member.

In the present exemplary embodiment, the first surface-treated layer 148 is not formed in the third supporting portion 143. In other words, in the third supporting portion 143, the surface of the first substrate 147a is exposed. As a result, the rigidity of the third supporting portion 143 is made less than that of an adjacent portion where the first surface-treated layer 148 is formed. In other words, the third supporting portion 143 has higher flexibility than the adjacent portion.

As illustrated in FIG. 3, the third supporting portion 143 is a portion that is to be subjected to bending at the tip of the finger 20 of the subject. According to the above configuration, not only the supporting member 14 can be bent with a smaller force, but also the maintainability of the bent state is enhanced. As a result, it is possible to suppress misalignment of the light emitter 11 and the light detector 12 after the sensor 10 is fixed to the finger 20. Therefore, it is possible to improve the convenience of the sensor 10 configured such that the light emitter 11 and the light detector 12 are attached to the finger 20 with the supporting member 14.

Although it is general to form the first surface-treated layer 148 on the surface of the first substrate 147 uniformly in order to provide a desired physical property, in the present exemplary embodiment, local omission of the first surface-treated layer 148 is intentionally performed in order to realize the enhancement of the local flexibility of the third supporting portion 143. Accordingly, the above-described effect can be obtained while reducing the cost associated with the formation of the first surface-treated layer 148.

However, it is not necessarily required to expose the surface of the first substrate 147a in the third supporting portion 143. As illustrated in FIG. 7, the first surface-treated layer 148 may be formed so as to cover the third supporting portion 143 as well. In this case, by providing another first surface-treated layer 148 in a portion adjacent to the third supporting portion 143, the flexibility of the third supporting portion 143 can be relatively enhanced.

As illustrated in FIG. 1, the first retaining portion 145 includes low adhesiveness portion 145a. The low adhesiveness portion 145a has lower adhesiveness than an adjacent portion.

As described with reference to FIGS. 4 and 5, when the sensor 10 is fixed to the finger, the second adhesive face 144d located on the back face side of the second retaining portion 146 is adhered to the surface of the first retaining portion 145. Since a portion having low adhesiveness is formed on the surface, the peelability of the second retaining portion 146 from the first retaining portion 145 can be enhanced. As a result, it is possible to facilitate the operation of re-attaching the sensor 10 in order to, for example, optimize the arrangement of the light emitter 11 and the light detector 12. Therefore, it is possible to improve the convenience of the sensor 10 configured such that the light emitter 11 and the light detector 12 are attached to the finger 20 with the supporting member 14.

In the present exemplary embodiment, similarly to the case of the third supporting portion 143 described with reference to FIG. 6, the formation of the first surface-treated layer 148 is locally omitted, whereby the low adhesiveness portion 145a is formed. In other words, the adhesiveness is relatively reduced by exposing the surface of the first substrate 147a having lower smoothness.

Although it is general to form the first surface-treated layer 148 on the surface of the first substrate 147 uniformly in order to provide a desired physical property, in the present exemplary embodiment, local omission of the first surface-treated layer 148 is intentionally performed in order to realize the low adhesiveness portion 145a. Accordingly, the above-described effect can be obtained while reducing the cost associated with the formation of the first surface-treated layer 148.

However, it is not necessarily required to expose the surface of the first substrate 147a in order to form the low adhesiveness portion 145a. Roughening may be performed on a portion of a first surface-treated layer 148 that is uniformly formed in order to cause the portion to have relatively low adhesiveness. Examples of roughening include printing, coating, adhesion of film members, and the like.

As an example, the first substrate 147a may be formed of a nonwoven fabric. In this case, since the first surface-treated layer 148 is formed in order to enhance the smoothness of the surface, not only the adhesiveness of the portion other than the low adhesiveness portion 145a can be enhanced, but also the fluffing on the surface of the nonwoven fabric can be suppressed. In order to suppress degradation of the smoothness of the surface, a woven fabric, a film material, a foam material, or the like may be used as the first substrate 147a.

As illustrated in FIG. 1, information for assisting attachment of the sensor 10 to the finger 20 is provided on the front face side of the supporting member 14. Such information includes information indicating the position of the light emitter 11, information indicating the position of the light detector 12, information providing a guide indicating the position at which the finger 20 is to be placed, and the like.

As illustrated in FIG. 6, the information is formed by a second surface-treated layer 149. The second surface-treated layer 149 may be formed on at least one of the surface of the first substrate 147a and the surface of the first surface-treated layer 148.

According to the above configuration, it is possible to improve the convenience of attachment of the sensor 10 to the finger 20, while ensuring the above-described advantage related to the high flexibility and the low adhesiveness.

In the present exemplary embodiment, the surface of the first surface-treated layer 148 is formed so as to exhibit a different appearance from of the surface of the first substrate 147a. As a result, the third supporting portion 143 and the low adhesiveness portion 145a on which the first surface-treated layer 148 is not formed have different appearance from other portions of the supporting member 14.

According to such a configuration, since it is possible to emphasize, to the user, a portion capable of providing the above-described advantage, it is possible to enhance the merchantability of the sensor 10.

The difference in appearance between the first substrate 147a and the first surface-treated layer 148 need not be provided by the difference in materials forming the respective elements. The difference in appearance may be provided by a color, a pattern, or the like.

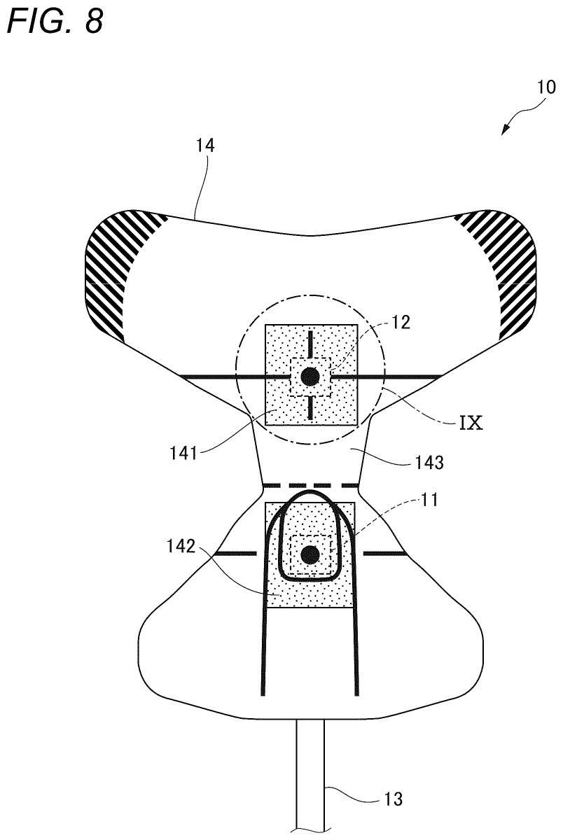

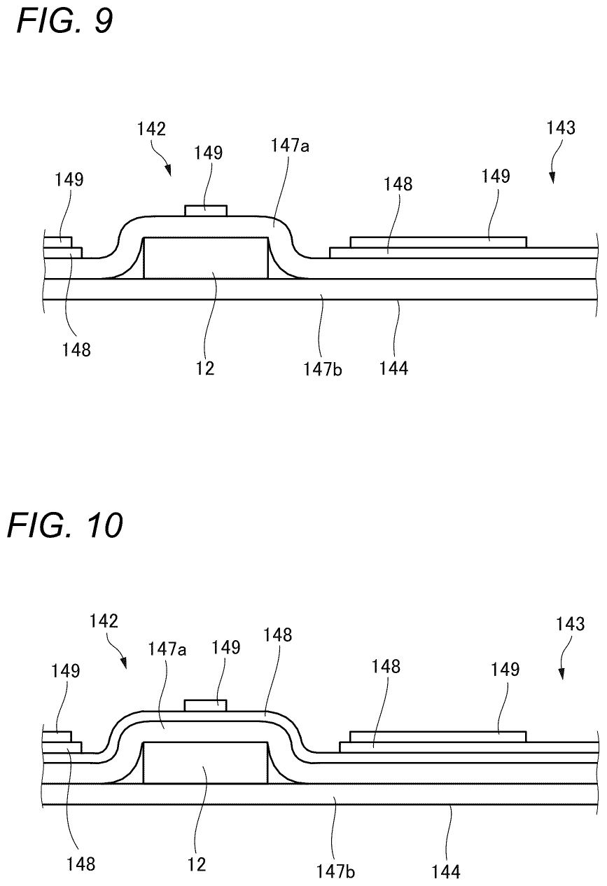

FIG. 8 illustrates another exemplary configuration of the sensor 10. FIG. 9 illustrates a sectional configuration of the supporting member 14 corresponding to a region IX illustrated in FIG. 8. In this example, the flexibility of each of the first supporting portion 141 and the second supporting portion 142 of the supporting member 14 is made higher than an adjacent portion.

Specifically, the first surface-treated layer 148 is not formed in the first supporting portion 141 and the second supporting portion 142 (the first supporting portion 141 is not illustrated). As a result, the rigidity of each of the first supporting portion 141 and the second supporting portion 142 is made less than that of the adjacent portion where the first surface-treated layer 148 is formed.

The first supporting portion 141 and the second supporting portion 142 are portions that support and cover the light emitter 11 and the light detector 12, respectively. According to such a configuration, elongation of the first substrate 147a due to the coverage of the light emitter 11 and the light detector 12 is promoted, so that it is possible to suppress generation of a stress as well as the misalignment of the light emitter 11 and the light detector 12 due to such stress. Therefore, it is possible to improve the convenience of the sensor 10 configured such that the light emitter 11 and the light detector 12 are attached to the finger 20 with the supporting member 14.

Although it is general to form the first surface-treated layer 148 on the surface of the first substrate 147 uniformly in order to provide a desired physical property, in the present exemplary embodiment, local omission of the first surface-treated layer 148 is intentionally performed in order to realize the enhancement of the local flexibility of the first supporting portion 141 and the second supporting portion 142. Accordingly, the above-described effect can be obtained while reducing the cost associated with the formation of the first surface-treated layer 148.

However, it is not necessarily required to expose the surface of the first substrate 147a in the first supporting portion 141 and the second supporting portion 142. As illustrated in FIG. 10, a first surface-treated layer 148 may be formed so as to cover the second supporting portion 142 as well (the same applies to the first supporting portion 141). In this case, by providing another first surface-treated layer 148 in a portion adjacent to each of the first supporting portion 141 and the second supporting portion 142, the flexibility of these portions can be relatively enhanced.

Each of the configurations exemplified above is merely illustrative for facilitating understanding of the present disclosure. The configuration examples may be appropriately modified or combined with other configuration examples without departing from the spirit of the present disclosure.

The portion in which the flexibility is made higher than the adjacent portion in the supporting member 14 may be at least one of the first supporting portion 141, the second supporting portion 142, and the third supporting portion 143, depending on the specification of the sensor 10.

As illustrated in FIG. 1, the supporting member 14 according to the above exemplary embodiment has a shape that is axisymmetric with respect to a straight line L connecting the light emitter 11 and the light detector 12. According to such a configuration, the attachment stability of the sensor with respect to the finger of the subject can be enhanced.

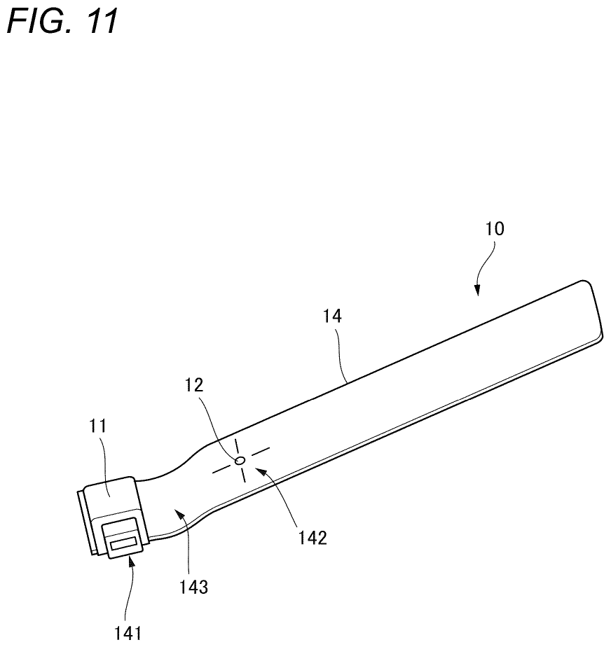

However, as illustrated in FIG. 11, the supporting member 14 may exhibit a belt shape in which the light emitter 11 is supported at one end thereof. In this example, fixation of the sensor 10 is performed by wrapping the supporting member 14 around the finger of the subject. The light detector 12 is supported at a position facing the light emitter 11 with the finger in between when the supporting member 14 is wrapped around the finger.

The application of the medical sensor to which the concept of the present disclosure may be applied is not limited to the detection of the blood light absorber concentration. In the case of a medical sensor that optically detects a physiological parameter, the fundamental concept of the present disclosure can be applied through an appropriate change of the shape.

1. A medical sensor, comprising:
at least one light emitter configured to emit light;
at least one light detector configured to output a signal corresponding to an amount of incident light; and
a supporting member adapted to be attached to a body of a subject,
wherein the supporting member includes:
a first supporting portion supporting and covering the light emitter;
a second supporting portion supporting and covering the light detector; and
a third supporting portion located between the first supporting portion and the second supporting portion; and
wherein at least one of the first supporting portion, the second supporting portion and the third supporting portion includes a portion with higher flexibility than an adjacent portion thereof.

2. The medical sensor according to claim 1,
wherein the supporting member includes:
a substrate; and
a first surface-treated layer formed on a surface of the substrate and having a different physical property from the substrate,
wherein the first surface-treated layer is not formed at the portion with higher flexibility.

3. The medical sensor according to claim 2,
wherein the first surface-treated layer is so formed as to exhibit a different appearance from the substrate.

4. The medical sensor according to claim 2,
wherein the supporting member includes a second surface-treated layer formed on a surface of at least one of the substrate and the first surface-treated layer so as to provide information for assisting attachment to the body of the subject.

5. The medical sensor according to claim 2,
wherein the substrate is formed with nonwoven fabric.

6. The medical sensor according to claim 1,
wherein the supporting member exhibits an axisymmetric shape relative to a straight line connecting the light emitter and the light detector.

7. A medical sensor, comprising:
at least one light emitter configured to emit light;
at least one light detector configured to output a signal corresponding to an amount of incident light; and
a supporting member adapted to be attached to a body of a subject,
wherein the supporting member includes:
a first retaining portion having a first adhesive face adapted to be adhered to the body; and
a second retaining portion having a second adhesive face adapted to be adhered to a surface of the first retaining portion while covering the surface,
wherein at least a part of the surface includes a portion with lower adhesiveness than an adjacent portion thereof.

8. The medical sensor according to claim 7,
wherein the supporting member includes:
a substrate; and
a first surface-treated layer formed on a surface of the substrate and having a different physical property from the substrate,
wherein the first surface-treated layer is not formed at the portion with lower adhesiveness.

9. The medical sensor according to claim 8,
wherein the first surface-treated layer is so formed as to exhibit a different appearance from the substrate.

10. The medical sensor according to claim 8,
wherein the supporting member includes a second surface-treated layer formed on a surface of at least one of the substrate and the first surface-treated layer so as to provide information for assisting attachment to the body of the subject.

11. The medical sensor according to claim 8,
wherein the substrate is formed with nonwoven fabric.

12. The medical sensor according to claim 7,
wherein the supporting member exhibits an axisymmetric shape relative to a straight line connecting the light emitter and the light detector.

13. A sensor supporting member adapted to attach, to a body of a subject, a medical sensor provided with at least one light emitter configured to emit light and at least one light detector configured to output a signal corresponding to an amount of incident light, the sensor supporting member comprising:
a first supporting portion supporting and covering the light emitter;
a second supporting portion supporting and covering the light detector; and
a third supporting portion located between the first supporting portion and the second supporting portion,
wherein at least one of the first supporting portion, the second supporting portion and the third supporting portion includes a portion with higher flexibility than an adjacent portion thereof.

14. A sensor supporting member adapted to attach, to a body of a subject, a medical sensor provided with at least one light emitter configured to emit light and at least one light detector configured to output a signal corresponding to an amount of incident light, the sensor supporting member comprising:
a first retaining portion having a first adhesive face adapted to be adhered to the body; and
a second retaining portion having a second adhesive face adapted to be adhered to a surface of the first retaining portion while covering the surface,
wherein at least a part of the surface includes a portion with lower adhesiveness than an adjacent portion thereof.
